(19) United States
(12) Patent Application Publication
(10) Pub. No.: US 2011/0172159 A1
(43) Pub. Date: Jul. 14, 2011

(54) PROTEIC ACID POLYMER, PRODUCTION PROCESSES, USE OF PROTEIC ACID POLYMER, PHARMACEUTICAL COMPOSITION AND METHOD OF TREATMENT

(76) Inventor: Tatiana Lobo Coelho de Sampaio, Rio de Janeiro (BR)

(21) Appl. No.: 13/062,322

(22) PCT Filed: Feb. 20, 2009
(86) PCT No.: PCT/BR2009/000051

§ 371 Date: Mar. 4, 2011

(30) Foreign Application Priority Data
Sep. 5, 2008 (BR) 020080116965

(51) Int. Cl. A61K 38/17; C07K 14/00; A61P 25/00; A61P 19/00; A61P 21/00; A61P 29/00

(52) U.S. Cl. 514/17.7; 530/395; 514/20.9

(57) ABSTRACT

The present invention relates to proteic acid polymers (pLNs) comprising specific properties to reduce tissue damage and improve functional recovery after injury, and the production process of said proteic acid polymers. Those pLNs are preferably obtained using the protein laminin diluted in an acidic pH in the presence of divalent cation. The use of said proteic acid polymers for the production of a drug, a pharmaceutical composition containing such pLNs and a method of treatment of animals affected by traumatic, degenerative or inflammatory tissue injuries in nervous tissue, muscle, epithelial and connective tissues in general are also objects of the present invention.

### FIELD OF THE INVENTION

The present invention relates to proteic acid polymer, the process of polymerization of a protein in acid medium and the use of said proteic polymer. Specifically, the polymerized protein described here is the polymerized laminin, extremely effective as promoter of tissue regeneration in humans or non-human mammals, due to its extraordinary anti-inflammatory effect.

The invention is also related to a pharmaceutical composition containing a proteic acid polymer, toward the treatment of humans or non-human mammals, affected by traumatic degenerative or inflammatory tissue injuries.

The use of such proteic acid polymer for the production of a drug intended mainly to the treatment of neurological, spinal cord, muscular dystrophy treatment and heart disease injuries is also an objective of this invention.

The use of such proteic acid polymer for the production of a drug intended mainly to the treatment of inflammation in nervous tissue, skeletal muscle, smooth muscle, cardiac muscle, the lining epithelium, adipose tissue, epithelial and connective tissues in general, also is an objective of this invention.

The invention also refers to a method of treatment of humans or non-humans mammals, affected by traumatic, degenerative or inflammatory injuries in the group of tissues including nervous tissue, skeletal muscle, smooth muscle, cardiac muscle, the lining epithelium, adipose tissue, epithelial and connective tissues in general, based on administration of a drug containing a proteic acid polymer in humans or non-human mammals.

Finally, the invention refers to a method of treatment for inflammatory diseases based on the administration of a drug containing a proteic acid polymer to humans or non-human mammals.

### BACKGROUND OF THE INVENTION

Laminin is an extracellular matrix protein originally described in the 70s that presents a trimeric structure, consisting of a longer chain, the alpha chain and two shorter chains, called beta and gamma. These three chains are associated through “coiled-coil” interactions resulting in a cross-shaped protein of approximately 800 KDa. This protein was originally purified from an extract of a murine tumor called EHS, which produces an excess of a laminar structure known as basement membrane (basal lamina). Later it was discovered that the protein was only the first member of a family of isoforms or a group of related proteins, which now has 16 members. The isolated protein from EHS is now known as laminin-1 (LN-1) and more recently as LN111. The LN-1 is primary expressed in embryonic tissues, but is also present in adult animals, but not in the nervous tissue itself. The expression of laminin in adult nervous tissue was studied in detail in 1989. The increase of its expression is significantly related to regenerative processes in invertebrates, in regions in the mammalian brain where axonal growth occurs during adulthood.

The U.S. Pat. No. 4,829,000, assigned to The United States of America represented by the Secretary of the Department of Health and Human Service and entitled “Reconstituted basement membrane complex with biological activity,” describes the production and use of an extract of basal membrane of mice, called Matrigel, from which laminin can be extracted.

The U.S. Pat. No. 6,632,790, assigned to University of Medicine and Dentistry of New Jersey and entitled “Laminin 2 and methods for its use”, describes the production and possible uses of recombinant human laminin 2, also called LN-2, and more recently LN-211. A point to be emphasized here is that the use of protein to treat spinal cord injury or inflammatory diseases was not anticipated in that patent. Moreover, there is no suggestion that the human recombinant laminin 2 as well as any other isoform of protein could be polymerized in acid pH.

The U.S. Pat. No. 5,019,087, assigned to American Biomaterials Corporation and entitled “Nerve regeneration conduit”, describes tubular prostheses for regeneration of peripheral nerves. The prostheses are made of collagen I or collagen I in addition to laminin. Interestingly, as the solubilization of collagen I requires acid pH, when laminin is added, the pH is already acid. This, however, is not done on purpose, but circumstantially.

The International application WO 03/035675, entitled “Biologically active peptides and their use for repairing injured nerves” describes small peptides derived from the sequence of laminin-1, containing the KDI tripeptide, which can promote the recovery of movement after a spinal cord transection. This patent describes the use of only a small fragment of the protein.

In 2000, Freire and Coelho-Sampaio showed that in the temperature of 35° C., LN-1 at low concentrations (between 5 nM to 60 nM) was able to self-polymerize provided that the solution was acidic, and more effectively, with a pH of 4.0. In this experiment it was essential to have the pre-treatment of the quartz cuvette, in which the laminin was contained, with non-adherent material such as silane. This treatment with silane was essential to maintain the laminin polymerized in its soluble state and not adhered to the quartz cuvette walls.

In 2002, Freire et al, showed that laminin polymers produced from adsorption on a glass surface in acidic pH (acid laminin matrix) played a role in migration and differentiation of embryonic neurons in cell culture or in cortical tissue explants, i.e. in vitro. This paper presents no test in vivo and makes no disclosure of other possible roles of such laminin polymers, such as for example, neural regeneration.

Thus, this invention seeks to fill all the gaps left in the state of the art, describing a new process, more efficient for polymerized laminin production in acid medium and employment of this polymerized protein in the treatment of spinal cord injury in animal models.

### SUMMARY OF THE INVENTION

It is an object of the invention, a proteic acid polymer that has anti-inflammatory and regenerative properties in traumatic, degenerative or inflammatory injuries of a tissue selected from the group of tissues comprising nervous tissue, skeletal muscle, smooth muscle, cardiac muscle, the lining epithelium, adipose tissue, epithelial and connective tissues in general, which in the presence of acid pH and a divalent cation polymerizes regardless of the presence of the cell membrane or other extracellular matrix components.

It is an additional object of this invention a process of production of proteic polymers in an acidic solution, whereas protein concentration ranges from 80 nM to 1 μM and the temperature, from 10 to 35° C. Polymers production is obtained in acid pH in the presence of a divalent cation, for a maximum incubation time of 12 hours and it takes place within containers produced with inert material, contaminant-free and without pre-treatment with anti-adherent substances.

Another object of the present invention concerns a pharmaceutical composition containing a pharmaceutically effective amount of a proteic acid polymer and non-active components, which are pharmaceutically acceptable.

It is still an object of this invention, the use of such proteic acid polymer for the production of a useful drug in the treatment of traumatic, degenerative or inflammatory injuries of a tissue selected from the group of tissues comprising nervous tissue, muscle tissue, smooth muscle, cardiac muscle, lining epithelium, adipose tissue, epithelial and connective tissues in general, such as the treatment of spinal cord and pulmonary injuries, muscular dystrophy and heart diseases in general, based on the application of a therapeutically effective amount of proteic polymers, on the injured region of an injured human or non-human mammal.

It is still an object of this invention, the use of such proteic acid polymer for production of a drug useful in the treatment of inflammation of a tissue selected from the group of tissues comprising nervous tissue, skeletal muscle tissue, smooth muscle, cardiac muscle, lining epithelium, adipose tissue, epithelial and connective tissues in general, based on the administration of a therapeutically effective amount of proteic polymer in an injured human or non-human mammal.

Another object of this invention is a method for the treatment of traumatic, degenerative or inflammatory injuries of a tissue selected from the group of tissues comprising nervous tissue, skeletal muscle, smooth muscle, cardiac muscle, lining epithelium, adipose tissue, epithelial and connective tissues in general based on the administration of a drug comprising a proteic acid polymer to a human or non-human mammal.

The last object of this invention is a treatment method of inflammation of a tissue selected from the group of tissues comprising nervous tissue, skeletal muscle, smooth muscle, cardiac muscle, lining epithelium, adipose tissue, epithelial and connective tissues in general, based on administration of a drug containing a proteic acid polymer to a human or non-human mammal.

### DESCRIPTION OF THE DRAWINGS

FIG. 1 shows a graph of BBB score versus weeks after injury, demonstrating that the laminin acid polymer (pLN) improves the performance of the open field locomotion test (BBB) after a spinal cord injury by compression. Functional recovery profiles observed after treatment with pLN (▪), compared to controls: laminin diluted in pH 7.0 (), acid buffer without protein (▾), neutral buffer without protein (▴).

FIG. 2 shows the recovery of the nervous tissue where the spinal cord injury by compression was induced, after application of the laminin acid polymer. In A—control treated with acid buffer only; in B—animal treated with laminin acid polymer. Both animals were euthanized 8 weeks after injury.

FIG. 3 shows the decrease in expression of GFAP protein promoted by treatment with laminin acid polymer. Panel A shows the area of the cystic cavity formed 8 weeks after injury in a control animal treated with acid buffer only. There is a high expression of GFAP. In B it is shown a decrease of the cavity area in animals treated with the laminin acid polymer, as well as a decrease of GFAP expression. Both animals tested 8 weeks after injury.

FIG. 4 shows neuronal regeneration as demonstrated by the expression of the protein, GAP-43, 8 weeks after injury. In A, control animals treated with acid buffer only; in B animals treated with laminin acid polymer.

FIG. 5 shows immunolabeling for macrophages (ED1 antibody), 8 weeks after injury. In A, control animals treated with acid buffer only, in B, animals treated with the laminin acid polymer. The result indicates that the effect of treatment with the laminin acid polymer reduces inflammation by decreasing macrophages infiltration in the injury region.

FIG. 6 shows the serum levels of C-reactive protein a week after injury. The analysis of the values obtained in each condition shows that treatment with laminin acid polymer promotes a reduction in systemic inflammation, supporting the hypothesis that the functional improvement observed in treated animals involves an anti-inflammatory effect of the polymer.

FIG. 7 shows the light scattering records of acid polymer samples formed by recombinant human LN-2 (continuous line) or control at pH 7 (dashed line).

FIG. 8 shows the BBB score versus weeks after transection injury. Functional recovery profiles are shown for: animals treated with PLN-1 (▴), polymers obtained from laminin purified from human placenta (▪) and PLN-2 (). Control shows the recovery achieved in the absence of laminin (V).

FIG. 9 shows the general anti-inflammatory effect of pLN in the body, giving the total number of cells in the bronchoalveolar wash of mice subjected to inhalation of LPS. A: LPS+acidic buffer; B: LPS+acidic laminin; C: LPS; D: Control.

### DETAILED DESCRIPTION OF THE INVENTION

This invention describes proteic acid polymer with anti-inflammatory and regenerative activity in traumatic, degenerative or inflammatory tissue injuries selected from the group of tissues comprising nervous tissue, skeletal muscle, smooth muscle, cardiac muscle, lining epithelium, adipose tissue, epithelial and connective tissues in general, being formed by polymerization of a particular protein in the presence of acid pH, a divalent cation and suitable temperature, being independent of the presence of cell membrane, the basement membrane and extracellular matrix components, being formed in vitro, in inert containers produced in any material, free of contaminants and without pre-treatment with anti-adherent substances.

The proteic acid polymers object of this invention are formed mainly by the interaction between the short arms of each molecule of laminin, which occurs without the cross linking between long and short arms of the laminin molecule.

The expression “injured region” should be understood as any animal organ or tissue formed by nervous tissue, skeletal muscle, smooth muscle, cardiac muscle, lining epithelium, adipose tissue, epithelial and connective tissues in general, including organs such as brain, spinal cord, muscles, heart, lungs and glands.

In nature, it is also verified the existence of laminin polymers, which form a proteic matrix. The formation of this matrix occurs without any cross interactions between long and short arms of laminin. The hindrance of such interactions is achieved in natural conditions when the long arms of laminin molecules interact with the specific receptor of cell membrane. Therefore, in natural conditions, it is essential that the laminin long arms anchor to the receptor located on the cell membrane in order to avoid the cross interaction between long and short arms of laminin adjacent molecules.

In the case of laminin polymers object of this invention that are formed in vitro, inhibition of cross reactions between long and short chains of molecules of adjacent laminin is due to medium acidification in which these polymers were formed. Thus the acidification of the medium mimics the action of the receptors of the cell membrane, which are absent in the object of this invention.

In tests performed here, it was verified that the tissue regenerative activity mediated by the proteic acid polymers developed by this invention, is increased when the application of these proteic acid polymers on the injured area occurs in a short period of time after injury.

The greater effectiveness of the proteic acid polymers, when applied onto the injury in a short period of time after the event of traumatic, degenerative or inflammatory injury is due to anti-inflammatory capacity promoted by proteic acid polymers of this invention. This anti-inflammatory ability occurs, because such proteic acid polymers act in the maintenance of basal serum levels of C-reactive protein, and in reducing the macrophages mobilization to the injured area. In tissues such as nerve tissue, this preservation is perceived by a reduction in the cystic cavity formation, a decrease in GFAP expression and the number of activated astrocytes in the damaged region. Another determinant factor of this tissue preservation is the reduction of natural inflammatory process that occurs at the injury site, demonstrated by a reduced macrophages infiltration and maintenance of basal serum levels of C-reactive protein. The latter effects induced by proteic acid polymers of this invention, make the site of injury more suitable for regeneration, e.g., the spinal cord and pulmonary parenchyma.

Besides, it was found that the proteic acid polymers of this invention, are able to promote regeneration, in the group of tissues comprising nervous tissue, skeletal muscle, smooth muscle, cardiac muscle, lining epithelium, adipose tissue, epithelial and connective tissues in general. This is based on the observation that there was an activation of the production of the growth-associated protein 43 (GAP43). It is well known that, in the nervous tissue this protein plays an important role in neurites formation, regeneration and plasticity.

Due to the mechanisms of action played by proteic acid polymer target of this invention, such polymers have an effective role, never before shown in promoting traumatic, degenerative or inflammatory injuries tissue regeneration in humans and non-human mammals.

The proteic acid polymer target of this invention can be useful in treatment of traumatic, degenerative or inflammatory injuries in a tissue selected from the group comprising nervous tissue, skeletal muscle, smooth muscle, cardiac muscle, lining epithelium, adipose tissue, epithelial and connective tissues in general. Preferably, the use of said proteic acid polymer is not restricted to spinal cord injury and pulmonary inflammation treatments. Due to the fact that such polymers induce neuroprotection and regeneration of nerve fibers, they can be used in the treatment of traumatic or degenerative injuries of the central and peripheral nervous system, where loss of nervous tissue may occur, and in treatment of muscular dystrophy and heart disease.

One advantage of proteic acid polymer described here is its ability to self-polymerize when in a medium with the appropriate acid pH. Moreover, it was demonstrated that the proteic acid polymer is able to restore the neuronal plasticity lost during development to newborn animals and to promote structural regeneration and functional recovery after spinal injury in adults.

It was also shown that the anti-inflammatory ability of the proteic acid polymer of this invention is systemic and not merely local, because it occurs in different body tissues, such as nervous and pulmonary tissues, and because it involves systemic reduction of serum levels of C-reactive protein.

The proteic acid polymer object of this invention is potentially the most effective therapeutic agent described to date to prevent degeneration and induce regeneration of spinal cord tissue and therefore allow the recovery of locomotion and sensitivity of treated animals.

Still preferably, these proteic acid polymers of this invention are effective therapeutic agents to combat respiratory tract and nervous system inflammatory injuries.

Preferably the proteic acid polymer object of this invention are laminin polymers made out of LN-1 from the EHS murine tumor, of recombinant human LN-2 or human LN extracted from placenta. Other protein sources can be used for production of these laminin polymers.

Henceforth, the proteic acid polymer will simply be called pLN, however, it should be said that this abbreviation should not mean in any case the limitation of the nature of these polymers only to the class of laminin proteins.

The production process of pLNs described here, beginning with the addition of a highly concentrated stock solution of a particular protein to be polymerized in a medium at acidic pH and containing a divalent cation, and should be done at temperature between 10° C. and 35° C., not requiring the pre-treatment of the container where the polymerization will occur with anti-adherent substances.

Besides the advantage to dispense the container pre-treatment with anti-adherent substances such as silane, where the production of pLNs will occur, the production process of such pLNs object of this invention is effective in promoting the polymerization at a broad range of concentrations. The protein concentration used in this process vary from 80 nM to 1000 μM. Preferably, the process is able to provide the polymerization of a protein in concentration from 90 nM to 500 μM; more preferably, from 95 nM to 300 μM.

Another factor that differentiates the pLNs production process object of this invention, with other protein polymerization processes in acid medium described above, is the fact that it occurs at room temperature, i.e. approximately 25° C., and is effective in pH from 3.0 and 6.0, occurring preferably at pH from 4.5 to 5.5. The acid solution used in this process, is any acid solution usually used in biochemistry, cell biology, cell culture or tissue culture or in animals in vivo.

The divalent cation required for the polymerization is preferably calcium, and all process of polymerization occurs at a maximum period of 12 hours, preferably the time required for the polymerization to be complete is, at maximum, 2 hours. More preferably, the process occurs in a maximum of 10 minutes.

The container required for the polymerization process object of this invention is an inert material container, free of contaminants and without pre-treatment with anti-adherents substances, such as silane. The container can be produced in any shape, among the shapes normally employed in the production of chemical, medical and pharmaceutical containers, with the material preferably employed being plastic, glass or quartz.

The pharmaceutical composition target object of this invention contains a pharmaceutically effective amount of pLN, object of this invention, and may contain non-active agents, such as adjuvants, stabilizers, solvents and lubricants. Preferably, the pharmaceutical composition contains a pharmaceutically effective amount of pLN, described by this invention.

In this invention, the term pharmaceutically effective amount is the amount of pNLs able to provoke a desirable effect on a human or non-human mammal.

Therefore, the pLNs target of this invention can be used in the production of a drug toward the treatment of traumatic, degenerative or inflammatory tissue injury, such tissue being selected from the group comprising nervous tissue, skeletal muscle, smooth muscle, cardiac muscle, lining epithelium, adipose tissue, epithelial and connective tissues in general, preferably, among the injuries that can be treated by a drug containing the pLNs, are spinal cord injury, muscular dystrophy, heart disease and lung injuries in humans or non-human mammals.

The pNLs can also be used in drug production aimed mainly to the treatment of inflammation in nervous tissue, skeletal muscle, smooth muscle, cardiac muscle, lining epithelium, adipose tissue, epithelial and connective tissues in general.

The drugs described in this invention can be prepared in any known pharmaceutical forms, provided that is allows the product application directly on the injured area.

The method of treatment subject of this invention can be used in the treatment of traumatic, degenerative or inflammatory tissue injury such tissue being selected from the group comprising nervous tissue, skeletal muscle, smooth muscle, cardiac muscle, lining epithelium, adipose tissue, epithelial and connective tissues in general, preferably, among the injuries that can be treated by a drug containing the pLNs, are spinal cord injury, muscular dystrophy, heart disease and lung injuries in humans or non-human mammals.

According to the treatment method of this invention, from 0.1 μg/kg to 1 mg/kg of a pLN should be applied directly on the site of spinal cord injury in a period of time less than 30 days after the lesion occurrence.

Preferably, from 0.5 to 500 μg/kg of a pLN should be applied directly on the injured region of a mammalian animal, and even more preferably, the treatment method of this invention requires the application directly on the injured region in mammals carrying a traumatic, degenerative or inflammatory injury in a amount from 1 to 250 μg/kg of a pLN, in a period of time lower than 15 days of the event of injury.

The following examples are related to tests conducted to prove the efficiency of the pLNs described, the production process of pLNs of this invention, as well as therapeutic clinical utility of such pLNs, being merely illustrative and should not be used in limiting the scope of the invention.

The tests were divided into three steps, as described below, during which were used two different types of pLNs: the pLN-1 corresponding to polymer obtained by the polymerization of protein extracted from a murine tumor EHS, and pLN-2—corresponding to the polymer obtained from recombinant human laminin expressed in mammalian cells.

### Example 1

### Production of pLNs

The first step of the description of the invention is the polymerization of laminin in acidic pH. The protein used can be the LN-1 extracted from the EHS murine tumor or recombinant human LN-2. The polymerization was made by diluting the protein in Tris-acetate buffer at concentration of 20 mM, pH about 4, containing 1 mM of calcium chloride, being calcium essential to the polymerization process. The protein previously aliquoted in volumes sufficient for animal treatment is removed from the freezer at −20° C., and kept on ice until the moment of injection in animals, which occurs after injury, preferably between 20 and 60 minutes after injury. The acetate buffer, maintained at room temperature (25° C.), is added to the aliquots of laminin present in a plastic tube (Eppendorf type), and homogenized with the pipette tip. The laminin final concentration varies between 50 to 200 μg/ml. About 5 to 10 μL of this suspension is injected into the animal preferably between 20 and 60 minutes after injury. In one assay LN-1 was used at 100 μg/ml, buffer pH 4.5, injected into the cord of the animal 30 minutes after the injury, and in another assay recombinant LN-2 at 120 μg/ml was used diluted in buffer pH 4.5, being injected 10 μL of suspension.

### Example 2

### Assays

In the example reported here an injury by compression between the eighth and ninth thoracic vertebrae (T8-T9), generated when a catheter placed between the cord and vertebra is inflated was used. The catheter was a Fogarty 2F and the volume of saline solution used to inflate the catheter was 15 μL. In this case, the untreated animal loses the proper functioning of the hind limbs, which is partially recovered over 8 weeks, but a cystic lesion within the cord remains, even after the animal has reached its best ability to move at the end of the eighth week (BBB=18). Surgery for catheter introduction was performed with the animal sedated with a cocktail of xilasine, acepromazine and ketamine for a laminectomy at the seventh thoracic vertebra (T7). After injury by compression the injections of LN-1 acid (pLN-1), pLN-2 acid (pLN-2), vehicles (acetate buffer pH 4 or tris buffer pH 7) were made. The injections were made locally (intratecal injections) as described above.

After treatment, 10 ml of Ringer's solution was injected for hidric and ionic restoration. Subsequently, the animals were treated with antibiotic (gentamicin sulfate), to reduce the chance of infections, mainly urinary, and analgesics, to minimize the sensation of pain due to surgery.

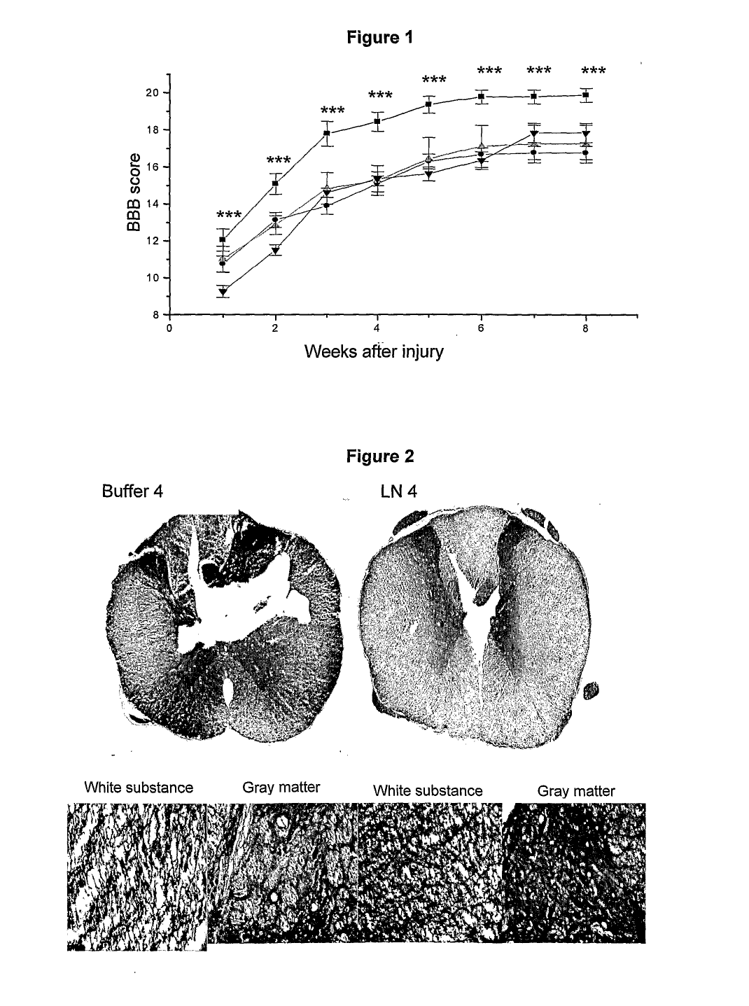

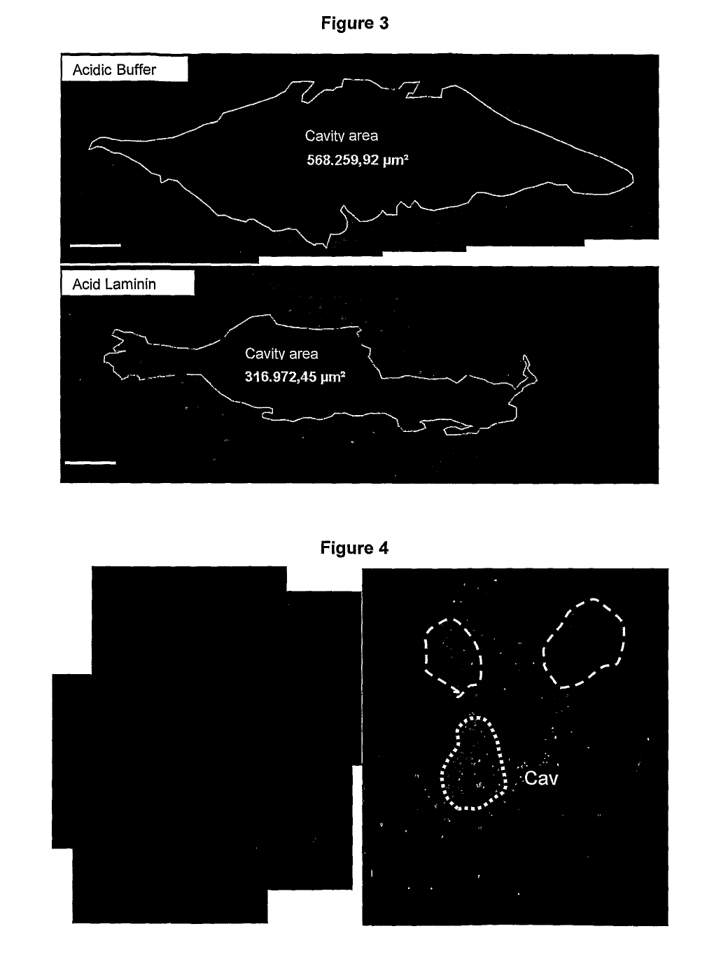

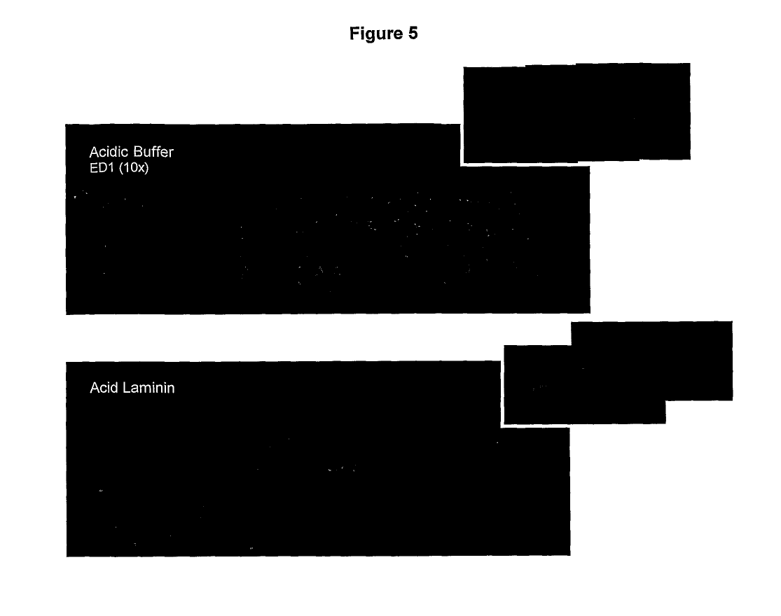

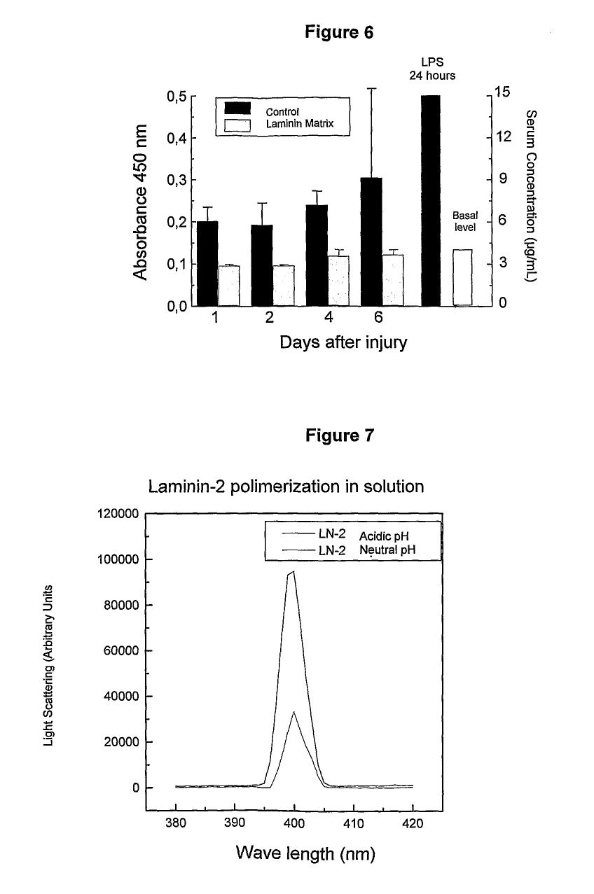

The monitoring of locomotor function of these animals through BBB scale (Basso, Beatie and Bresnahan) revealed that the animals treated with pLN had a functional recovery much faster than those treated with vehicle. FIG. 1 show that in 6 weeks the treated animals received a grade of BBB of 20, which corresponds to an almost normal locomotion, while the untreated animals had a maximum score of 17 after 8 weeks. Morphological analysis of tissue recovery shows that while the control animals (not treated with pLN) had a large cystic cavity at the site of injury, treated animals had a tissue morphologically more organized, as seen in FIG. 2.

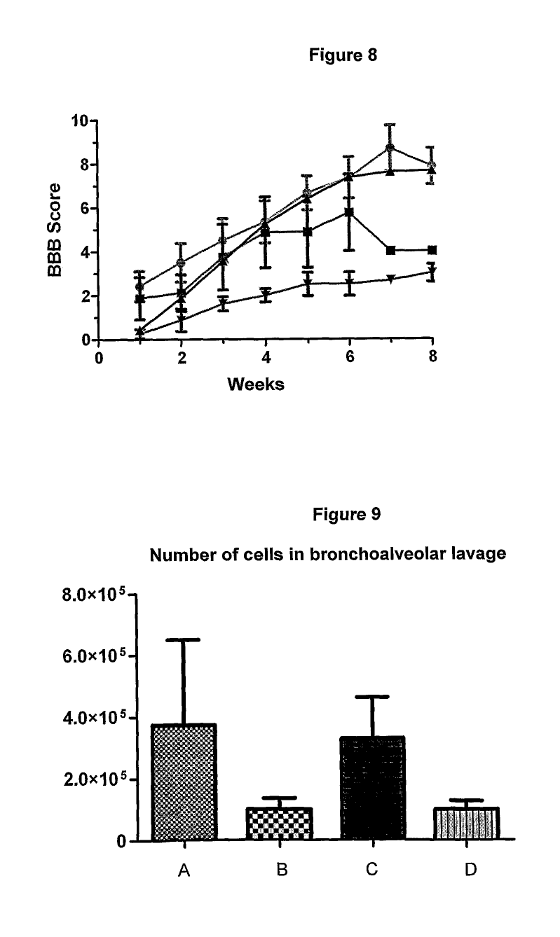

The results documented in this invention were obtained with LN-1 extracted and purified from EHS murine tumor. As the use of a protein obtained from animal source can lead to the transfer and adaptation of animal viruses, it was proposed that human laminin should be used in therapies to human patients. Similar results to those presented here were obtained with recombinant human LN-2 and are shown in FIG. 8.

### Example 3

### Application of Laminin

The application of laminin was made through a manually controlled injection so that approximately 1 μL penetrated the tissue every minute. The site was exactly the site of compression or in the proximal area of spinal cord in relation to the injury, in the case of trans-section. The injection syringe used was a Hamilton 80330 for 10 μL.

This invention is a solution for the treatment of spinal cord injuries being innovative mainly because therapeutic strategies for this type of injury does not exist. The conventional treatment currently available aims to stop the progression of initial damage, i.e., it tries to reduce the inflammatory reaction and secondary tissue damage. Conventional treatments promote surgical stabilization of the spine and treats patients with methylprednisolone, which does not result in consistent benefit for the patient. The laminin polymerized in acid pH proposed here can induce the regeneration of nerve tissue injured in a compression, contusion or transection of the spinal cord.

1. Proteic acid polymers comprising a regenerative and an anti-inflammatory activity, being formed by polymerization of a protein in the presence of acidic pH medium and a divalent cation.

2. Polymers according to claim 1 wherein said polymerized protein is preferably a laminin.

3. Polymers according to claim 2 wherein the polymerized laminin may comprise: the LN-1 from EHS murine tumor, recombinant human LN-2, laminin extracted from human placenta, or a combination thereof.

4. Polymers according to claim 1, which have anti-inflammatory and regenerative properties in traumatic, degenerative or inflammatory injuries in a tissue selected from the group comprising the nervous tissue, skeletal muscle, smooth muscle, cardiac muscle, the lining epithelium, adipose tissue, epithelium-conjunctive in general and combination thereof.

5. Process for the production of proteic acid polymers comprising the step of adding a high concentration of a particular protein to be polymerized in a acidic pH medium containing a divalent cation, performed at temperature between 10° C. and 35° C. for a maximum time of 12 hours.

6. Process according to claim 5 in which the temperature of reaction is approximately 25° C., and the acidic medium has a pH between 3.0 and 6.0.

7. Process according to claim 5 in which calcium is the divalent cation.

8. Process according to claim 5 in which polymerization occurs to a maximum of 2 hours.

9. Process according to claim 6 comprising a pH in the range between 4.0 and 5.5.

10. Process according to claim 5 comprising the ability to polymerize proteins in a concentration between 80 nM to 1000 μM.

11. Process according to claim 10 comprising the ability to polymerize proteins in a concentration between 90 nM to 500 μM.

12. Process according to claim 11 comprising the ability to polymerize proteins in a concentration between 95 nM to 300 μM.

13. Pharmaceutical composition comprising an pharmaceutically effective amount of a proteic acid polymers and non-active components.

14. Composition according to claim 13 comprising a pharmaceutically acceptable amount of a laminin proteic acid polymers.

15. Method for treatment of traumatic, degenerative or inflammatory tissue injuries in the group of tissues comprising nervous tissue, skeletal muscle, smooth muscle, cardiac muscle, the lining epithelium, adipose tissue and epithelium-conjunctive and combinations thereof comprising the application of a therapeutically effective amount of a proteic acid polymers in an mammal.

16. Method of treatment according to claim 15 comprising administering of an amount between 0.1 μg/Kg to 1 ng/Kg of a proteic acid polymers on traumatic, inflammatory or degenerative tissue injuries, such as spinal cord injury, muscular dystrophy, heart and lung injuries of a human or non human mammals.

17. Method of treatment according to claim 16 comprising administering of an amount between 0.5 μg/Kg to 500 μg/Kg of a proteic acid polymers directly on the injured area.

18. Method of treatment according to claim 17 comprising administering of an amount between 1 to 250 μg/Kg of a proteic acid polymers on traumatic, degenerative or inflammatory tissue injuries in a human or non human mammal.

19. Use of proteic acid polymers in the manufacture of a drug for the treatment of traumatic, degenerative or inflammatory injuries in the group of tissues selected from the group of nervous tissue, skeletal muscle, smooth muscle, cardiac muscle, the lining epithelium, adipose tissue and epithelium-conjunctive and combinations thereof.

20. Use of proteic acid polymers in the manufacture of a drug for the treatment of inflammation in nervous tissue, skeletal muscle, smooth muscle, cardiac muscle, the lining epithelium, adipose tissue, epithelium-conjunctive in general and combinations thereof.
